(19) United States
(12) Patent Application Publication
(10) Pub. No.: US 2026/0166002 A1
(43) Pub. Date: Jun. 18, 2026

(54) PHARMACEUTICAL COMPOSITION FOR TREATMENT OF PROSTATE CANCER OR BREAST CANCER

(71) Applicant: TOKYO METROPOLITAN GERIATRIC HOSPITAL AND INSTITUTE OF GERONTOLOGY, Tokyo (JP)

(72) Inventors: Satoshi INOUE, Tokyo (JP); Ken-ichi TAKAYAMA, Tokyo (JP); Hiroyuki OSADA, Saitama (JP); Yasumitsu KONDOH, Saitama (JP)

(21) Appl. No.: 19/531,003

(22) Filed: Feb. 5, 2026

Related U.S. Application Data
(63) Continuation-in-part of application No. 18/246,522.

(30) Foreign Application Priority Data
Sep. 30, 2020 (JP) 2020-165745

(51) Int. Cl. A61K 31/366; A61K 31/404; A61K 31/453; A61K 31/5365; A61K 31/5377; A61P 35/00

(52) U.S. Cl. CPC A61K 31/366; A61K 31/404; A61K 31/453; A61K 31/5365; A61K 31/5377; A61P 35/00

(57) ABSTRACT

The object of the present invention is to provide a therapeutic agent for treating hormone therapy-resistant, treatment-resistant cancers, and a method of the treatment of hormone therapy-resistant, treatment-resistant cancers. The problem can be solved by a pharmaceutical composition including a compound represented by the formula (1) or a salt thereof or a compound represented by the formula (2) or (3) or a salt thereof, as an active ingredient for treating prostate cancer. Furthermore, the pharmaceutical composition of the present invention can effectively treat not only hormone therapy-resistant prostate cancer or breast cancer but also non-hormone therapy-resistant prostate cancer or breast cancer and Castration-resistant prostate cancer or treatment-resistant breast cancer caused by other mechanisms.

### CROSS REFERENCE TO RELATED APPLICATIONS

This application is a Continuation-in-Part application of U.S. application Ser. No. 18/246,522, filed Mar. 24, 2023. U.S. application Ser. No. 18/246,522 is a U.S. National Phase filing under 35 U.S.C. § 371 of International Application PCT/JP2021/015982, filed Apr. 20, 2021, and published as WO 2022/070483 A1 on Apr. 7, 2022. PCT/JP2021/015982 claims priority from Japanese application number 2020-165745, filed Sep. 30, 2020. The entire contents of each of these prior applications are hereby incorporated herein by reference.

### TECHNICAL FIELD

The present invention relates to a pharmaceutical composition for treating the prostate cancer or breast cancer. According to the present invention, refractory prostate cancer or breast cancer can be treated.

### BACKGROUND ART

Prostate cancer and breast cancer are the most common cancers affecting men and women in Europe and the United States. In Japan, the number of patients with prostate cancer is increasing dramatically due to the Westernisation of the diet and the ageing of the population. The growth of prostate cancer is generally stimulated by androgen, which is a male hormone. On the other hand, estrogen, which is a female hormone, stimulates the growth of breast cancer. Therefore, hormone therapies that inhibit androgen and estrogen production and function are usually used for the treatment of prostate cancer and breast cancer. Although the effect of hormone therapy is initially excellent, prostate cancer relapses within a few years as castration-resistant prostate cancer (CRPC). In addition, the acquisition of resistance to hormone therapy is also a problem in breast cancer (PATENT LITERATURES 1 and 2). Thus, the control of treatment-resistant cancers that are resistant to hormone therapy is the most important problem to be solved.

### CITATION LIST

### Patent Literature

[PATENT LITERATURE 1] WO 2014/188775
[PATENT LITERATURE 2] JP 2013-17414 A

### SUMMARY OF INVENTION

### Technical Problem

Therefore, the object of the present invention is to provide a therapeutic agent for treating hormone therapy-resistant, treatment-resistant cancers, and a method of the treatment of hormone therapy-resistant, treatment-resistant cancers.

### Solution to Problem

The inventors have studied intensively on therapeutic agents and methods for treating hormone therapy-resistant, treatment-resistant cancers. Surprisingly, they found that two groups of compounds with specific chemical structures can effectively treat hormone therapy-resistant, treatment-resistant cancers.

The present invention is based on these findings.

Therefore, the present invention relates to
[1] A pharmaceutical composition for treating prostate cancer comprising:
a compound represented by a following formula (1):

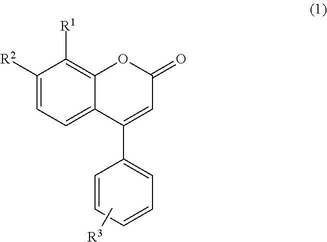

wherein R¹ is a hydrogen atom, a hydroxyl group, an alkyl group having 1 to 6 carbon atoms, an alkoxy group having 1 to 6 carbon atoms, or a heterocyclic alkyl group, and
R² is a hydrogen atom, a hydroxyl group, an alkyl group having 1 to 6 carbon atoms, or an alkoxy group having 1 to 6 carbon atoms, or R¹ and R² together form a 5 to 7 membered heterocyclic ring, which may have a substituent, said substituent being a hydroxyl group, an alkyl group having 1 to 6 carbon atoms, an alkoxy group having 1 to 6 carbon atoms, or a phenylalkyl group having 6 to 12 carbon atoms,
R³ is a hydrogen atom, a hydroxyl group, an alkyl group having 1 to 6 carbon atoms, or an alkoxy group having 1 to 6 carbon atoms, or a salt thereof, or
a compound represented by a following formula (2) or (3):

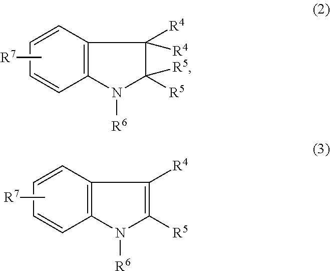

wherein R⁴ is each independently a hydrogen atom, an aldehyde group, a hydroxy group, an alkyl group having 1 to 6 carbon atoms, or an alkyl ester alkylene group, or two
R⁴ together are an oxygen atom,
R⁵ is each independently a hydrogen atom, an aldehyde group, a hydroxy group, an alkyl group having 1 to 6 carbon atoms, an alkyl ester group having 1 to 6 carbon atoms, or an alkyl ester alkylene group, or two R⁵ together are an oxygen atom,
R⁶ is a hydrogen atom or an alkyl group having 1 to 6 carbon atoms, and
R⁷ is a hydrogen atom, a hydroxyl group, an alkyl group having 1 to 6 carbon atoms,
an alkyl ester group having 1 to 6 carbon atoms, or an alkoxy group having 1 to 6 carbon atoms, or a salt thereof, as an active ingredient.

[2] The pharmaceutical composition for treating prostate cancer according to [1], wherein the compound represented by the formula (1) is a compound selected from the group consisting of

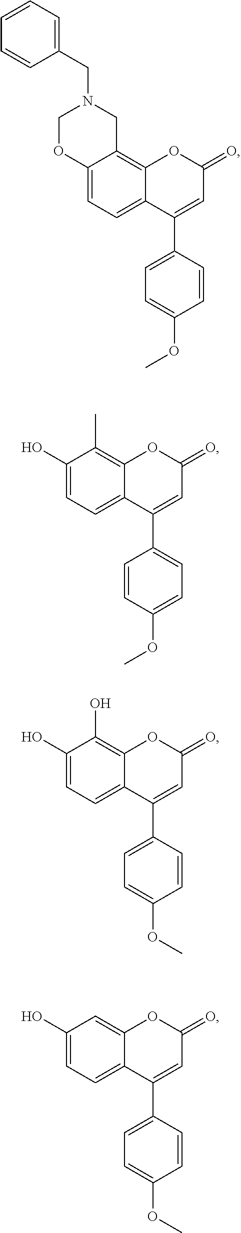 [SMILES: COc1ccc(-c2cc(=O)oc3c4c(ccc23)OCN(Cc2ccccc2)C4)cc1 ; COc1ccc(-c2cc(=O)oc3c(C)c(O)ccc23)cc1 ; COc1ccc(-c2cc(=O)oc3c(O)c(O)ccc23)cc1 ; COc1ccc(-c2cc(=O)oc3cc(O)ccc23)cc1]

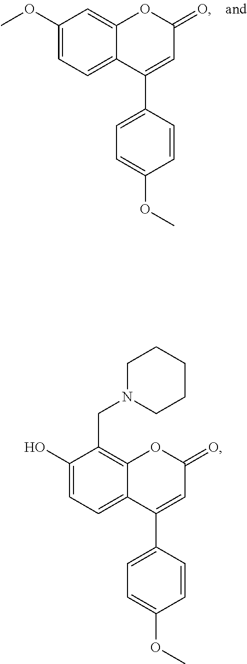 [SMILES: COc1ccc(-c2cc(=O)oc3cc(OC)ccc23)cc1 ; COc1ccc(-c2cc(=O)oc3c(CN4CCCCC4)c(O)ccc23)cc1]

the compound represented by the formula (2) is a compound selected from the group consisting of

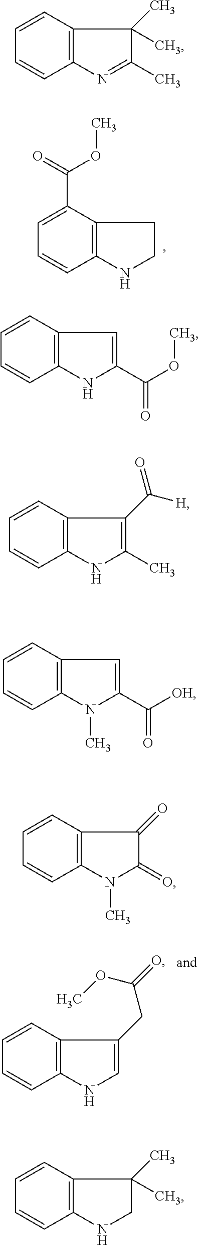

[3] A pharmaceutical composition for treating breast cancer comprising:
a compound represented by a following formula (1):

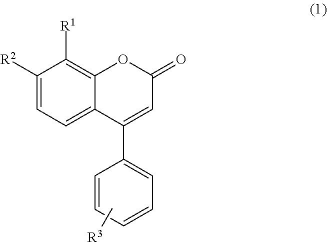

wherein R¹ is a hydrogen atom, a hydroxyl group, an alkyl group having 1 to 6 carbon atoms, an alkoxy group having 1 to 6 carbon atoms, or a heterocyclic alkyl group, and
R² is a hydrogen atom, a hydroxyl group, an alkyl group having 1 to 6 carbon atoms, or an alkoxy group having 1 to 6 carbon atoms, or R¹ and R² together form a 5 to 7 membered heterocyclic ring, which may have a substituent, said substituent being a hydroxyl group, an alkyl group having 1 to 6 carbon atoms, an alkoxy group having 1 to 6 carbon atoms, or a phenylalkyl group having 6 to 12 carbon atoms,
R³ is a hydrogen atom, a hydroxyl group, an alkyl group having 1 to 6 carbon atoms, or an alkoxy group having 1 to 6 carbon atoms, or a salt thereof, or a compound represented by a following formula (2) or (3):

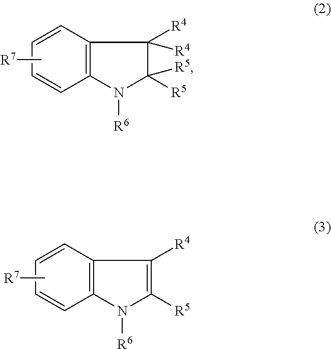

wherein R⁴ is each independently a hydrogen atom, an aldehyde group, a hydroxy group, an alkyl group having 1 to 6 carbon atoms, or an alkyl ester alkylene group, or two
R⁴ together are an oxygen atom,
R⁵ is each independently a hydrogen atom, an aldehyde group, a hydroxy group, an alkyl group having 1 to 6 carbon atoms, an alkyl ester group having 1 to 6 carbon atoms, or an alkyl ester alkylene group, or two R⁵ together are an oxygen atom,
R⁶ is a hydrogen atom or an alkyl group having 1 to 6 carbon atoms, and
R⁷ is a hydrogen atom, a hydroxyl group, an alkyl group having 1 to 6 carbon atoms, an alkyl ester group having 1 to 6 carbon atoms, or an alkoxy group having 1 to 6 carbon atoms, or a salt thereof, as an active ingredient, and

[4] The pharmaceutical composition for treating breast cancer according to [3], wherein the compound represented by the formula (1) is a compound selected from the group consisting of

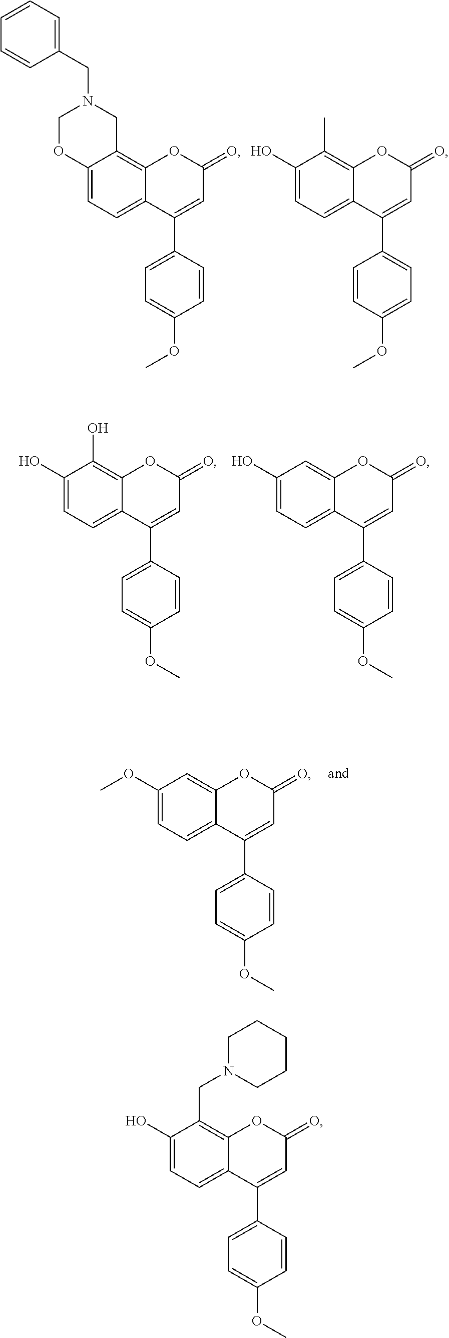

the compound represented by the formula (2) is a compound selected from the group consisting of

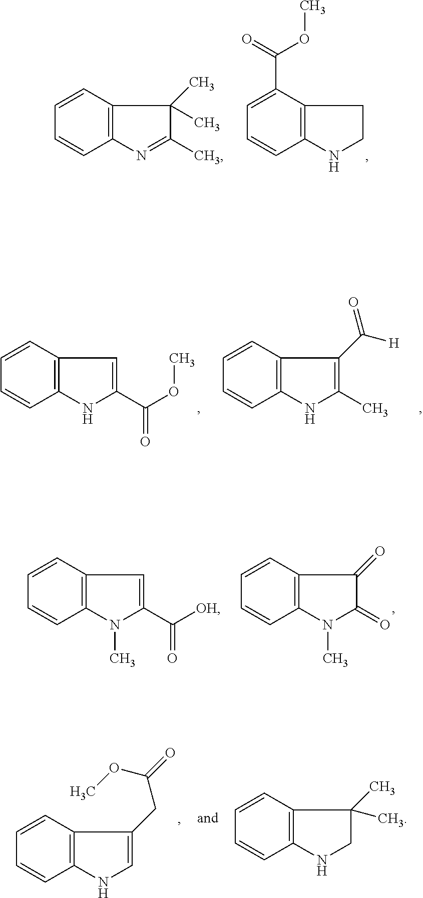

[5] The pharmaceutical composition for treating prostate cancer according to [1], wherein the compound represented by the formula (1) is the following compound:

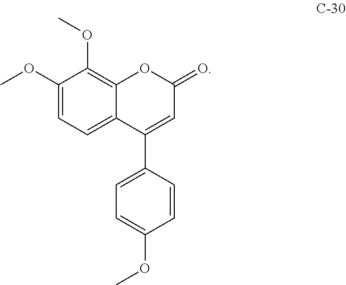 [SMILES: COc1ccc(-c2cc(=O)oc3c(OC)c(OC)ccc23)cc1]

[6] The pharmaceutical composition for treating breast cancer according to [3], wherein the compound represented by the formula (1) is the following compound:

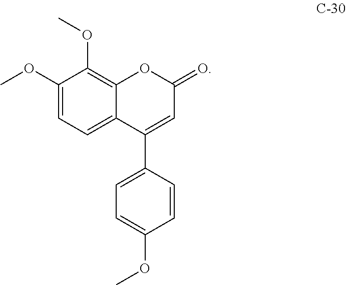 [SMILES: COc1ccc(-c2cc(=O)oc3c(OC)c(OC)ccc23)cc1]

[7] A pharmaceutical composition for treating prostate cancer or breast cancer comprising a compound represented by any one of the following formulae:

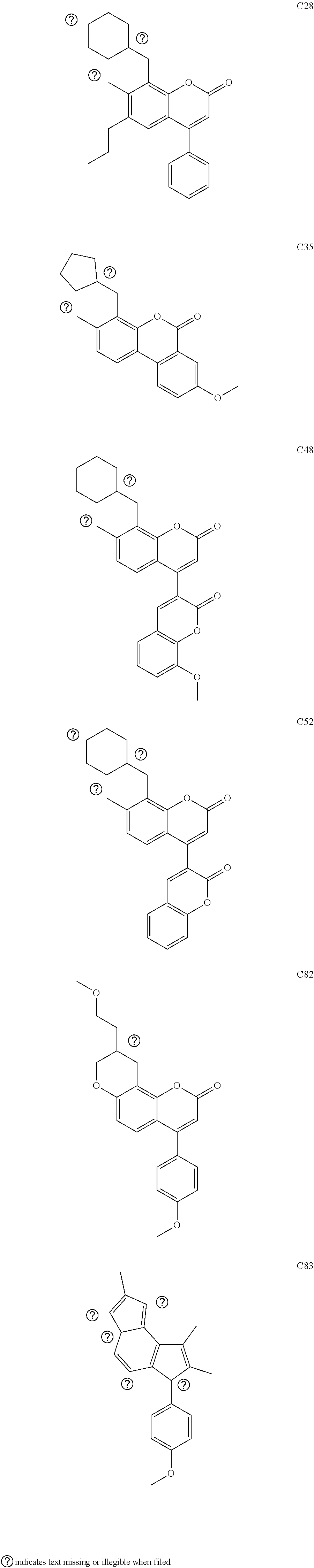

or a salt thereof, as an active ingredient.

### Advantageous Effects of Invention

According to the pharmaceutical composition of the present invention, hormone therapy-resistant prostate cancer or hormone therapy-resistant breast cancer can be effectively treated. Furthermore, the pharmaceutical composition of the present invention can effectively treat not only hormone therapy-resistant prostate cancer or breast cancer but also non-hormone therapy-resistant prostate or breast cancer and Castration-resistant prostate cancer or treatment-resistant breast cancer caused by other mechanisms.

### BRIEF DESCRIPTION OF THE DRAWINGS

FIG. 1 is a graph showing the proliferation inhibitory effects of compounds 10-0, 10-1, 10-3, 14-0, and 14-5 on hormone therapy-resistant prostate cancer model cell line 22Rv1, hormone therapy-resistant model LTAD (long time androgen deprived) cells, and VCaP cells.

FIG. 2 is a graph showing the result of the measurement of the inhibitory capacity of compounds 10-0, 10-1, 10-2, 10-3, 10-4, 14-0, and 14-5 to PSF interaction with its target RNA by the RNA pull-down method.

FIG. 3 is a graph showing the cell proliferation inhibitory effects of compounds 10-0, 10-1, 10-3, 10-4, 14-0, and 14-5 using 22Rv1 cells (prostate cancer), MCF7 cells (breast cancer), and OHTR cells (breast cancer).

FIG. 4 is a graph showing the inhibition of expression of the target genes of PSF, which are AR, AR-V7, SchLaP1, FKBP5, and ACSL3, in 22Rv1 cells (prostate cancer), by compounds 10, 10-1, and 10-3.

FIG. 5 is a graph showing the inhibition of binding of compounds 10, 10-1, and 10-3 to the mRNAs of ERα, SCFD2, and TRA2B, which are the target genes of PSF, and GAPDH (control), in OHT-TamR cells (breast cancer).

FIG. 6 is a diagram showing the results of examining the in vivo anticancer effects of compound 10-3 using a mouse model in which AR-positive CRPC tumors (22Rv1 cells) are subcutaneously transplanted.

FIG. 7 is a graph and photographs showing the results of examining the expression of Ki67 and AR in tumor tissues treated with compound 10-3.

FIG. 8 is a graph showing the cell proliferation inhibitory effects of compounds 10-1 and 10-3 using a model cell (DU145 cells) of Castration-resistant prostate cancer, which is AR-negative and has a mutation of tumor suppressor p53.

FIG. 9 is a diagram showing the in vivo anticancer effects of compound 10-3 using a mouse model in which AR-negative CRPC tumors (DU145 cells) are subcutaneously transplanted.

FIG. 10 is a diagram showing the results of examining the binding of the three PSF mutants (mut #1: K516I, D517V, mut #2: Y490H, mut #1+2: Y490H, K516I, D517V) and wild-type PSF to the RNA strand of CTBP1-AS, confirmed by RNA pull-down method. Docking analysis to determine the binding site of compound 10-3 to its target protein, PSF, suggested that the position at which each mutation was introduced interacted with compound 10-3.

FIG. 11 shows that a novel PSF inhibitor, C-30, was identified through a screening test based on similarity to the previously identified PSF inhibitor No. 10-3. (A) Screening procedure for optimized small-molecule compounds. A computer-aided selection procedure was applied to identify 50 compounds similar to No. 10-3. (B) Structure of No. 10-3 and optimized compound C-30. (C) Comparison of the inhibitory activities of No. 10-3 and C-30 using RNA pulldown assay. In vitro inhibition of the interaction between PSF and RNA was examined using 22Rv1 and biotin-labeled CTBP1-AS RNA probes. (D) The effects of No. 10-3 and C-30 were evaluated by estimating their IC50 values. The quantified protein expression of PSF obtained by RNA pull-down (N=3, biological replicates) is plotted.

FIG. 12 is a diagram showing the seven small compounds for the screening test and results the screening test. (A) Compounds were selected with drug-likeness properties by computer-aid. Structures of seven compounds similar to No. 10 or No. 10-3, which repressed breast (OHTR) and prostate cancer (22Rv1) cell proliferation, were shown. (B)

Second screening step using the MTS assay. The viability of 22Rv1 and OHTR cells treated with 1 or 5 μM of each compound for 72h. Two-sided Student's t-test was performed to analyze the differences. Ct: Vehicle control.

FIG. 13 is a graph showing inhibition of hormone therapy-resistant prostate and breast cancer cell proliferation by No. 10-3 and C-30. 4-hydroxytamoxifen-resistant (OHTR) and 22Rv1 cells were incubated with No. 10-3 or C-30 at the indicated concentrations for three days (N=3). *P<0.05, **P<0.01 between C-30 and No. 10-3 as determined by a two-sided student t-test. Ct: vehicle control.

FIG. 14 is a graph showing improvement of anti-cancer activity by C-30 against hormone receptor-negative cancer cells with mutated p53. Cell viability was determined using the MTS assay in DU145 (N=4), PC3 (N=3), and MDA-MB-231 (N=4) cells harboring p53 mutations treated with No. 10-3 and C-30 (biological replicates). The IC50 of No. 10-3 and C-30 in repressing cell viability are shown on the right (No. 10-3) or left (C-30). A two-sided t-test was performed to determine the statistical difference between No. 10-3 and C-30. *: P<0.05, **: P<0.01, ***: P<0.001.

FIG. 15 is a graph showing that C-30 represses tumor growth in p53-mutated cancer cell xenograft model. Nude mice (male) were inoculated with the DU145 cells. Nude mice (female) were inoculated with MDA-MB231 cells. The mice were treated with vehicle (control), No. 10-3 or C-30 (10 mg/kg three times per week). Tumor growth in the nude mice is shown (N=6; biologically independent animals). One-way analysis of variance (ANOVA) was performed, followed by two-sided t-tests. Data are presented as the mean±S.D.

### DESCRIPTION OF EMBODIMENTS

The pharmaceutical composition for treating prostate cancer or the pharmaceutical composition for treating breast cancer of the present invention comprises: a compound (hereinafter referred to as compound A) represented by a following formula (1):

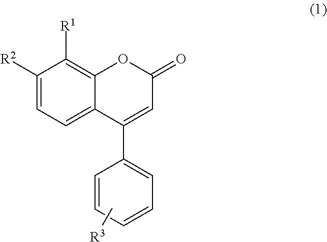

wherein R¹ is a hydrogen atom, a hydroxyl group, an alkyl group having 1 to 6 carbon atoms, an alkoxy group having 1 to 6 carbon atoms, or a heterocyclic alkyl group, and
R² is a hydrogen atom, a hydroxyl group, an alkyl group having 1 to 6 carbon atoms, or an alkoxy group having 1 to 6 carbon atoms, or R¹ and R² together form a 5 to 7 membered heterocyclic ring, which may have a substituent, said substituent being a hydroxyl group, an alkyl group having 1 to 6 carbon atoms, an alkoxy group having 1 to 6 carbon atoms, or a phenylalkyl group having 6 to 12 carbon atoms,
R³ is a hydrogen atom, a hydroxyl group, an alkyl group having 1 to 6 carbon atoms, or an alkoxy group having 1 to 6 carbon atoms, or a salt thereof, or
a compound (hereinafter referred to as compound B) represented by a following formula (2) or (3):

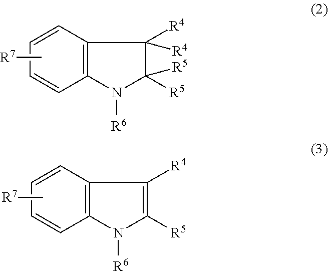

wherein R⁴ is each independently a hydrogen atom, an aldehyde group, a hydroxy group, an alkyl group having 1 to 6 carbon atoms, or an alkyl ester alkylene group, or two R⁴ together are an oxygen atom,
R⁵ is each independently a hydrogen atom, an aldehyde group, a hydroxy group, an alkyl group having 1 to 6 carbon atoms, an alkyl ester group having 1 to 6 carbon atoms, or an alkyl ester alkylene group, or two R⁵ together are an oxygen atom,
R⁶ is a hydrogen atom or an alkyl group having 1 to 6 carbon atoms, and
R⁷ is a hydrogen atom, a hydroxyl group, an alkyl group having 1 to 6 carbon atoms, an alkyl ester group having 1 to 6 carbon atoms, or an alkoxy group having 1 to 6 carbon atoms, or a salt thereof, as an active ingredient, or
a compound (hereinafter referred to as compound C) represented by any one of the following formulae:

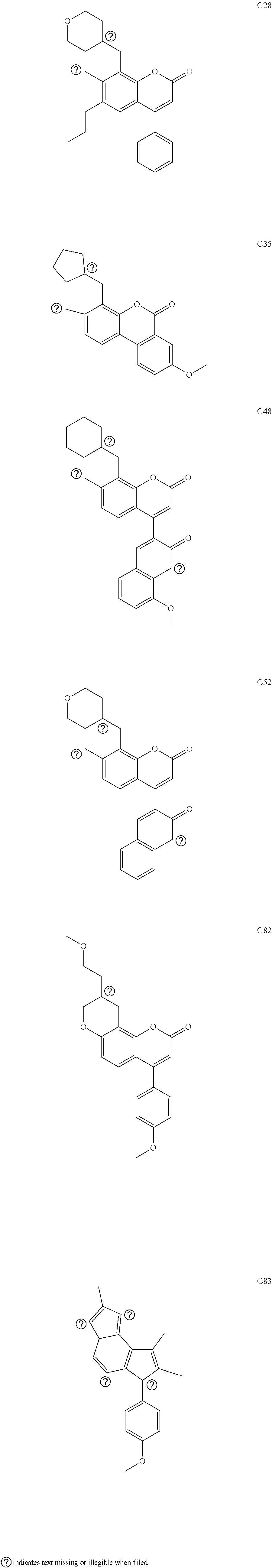

or a salt thereof, as an active ingredient.

Specifically, the examples of an alkyl group having 1 to 6 carbon atoms include a methyl group, an ethyl group, a normal propyl group, an isopropyl group, a normal butyl group, an isobutyl group, a secondary butyl group, a tertiary butyl group, a normal pentyl group, an isopentyl group, a neopentyl group, a tertiary pentyl group, a normal hexyl group, and an isohexyl group. An alkyl group having 1 to 3 carbon atoms is preferred.

Specifically, the examples of an alkoxy group having 1 to 6 carbon atoms include a methoxy group, an ethoxy group, a normal propoxy group, an isopropoxy group, a normal butoxy group, an isobutoxy group, a secondary butoxy group, a tertiary butoxy group, a normal pentyloxy group, an isopentyloxy group, a neopentyloxy group, a normalhexyloxy group, or an isohexyloxy group. An alkoxy group having 1 to 3 carbon atoms is preferred.

A heterocyclic alkyl group is a heterocycle group (a monovalent group formed by detaching one hydrogen atom attached to the ring of a heterocyclic compound) to which an alkylene group is attached. A heterocycle group is, but not limited to, preferably a saturated or unsaturated 5- or 6-membered ring, more preferably a saturated 5- or 6-membered ring, such as pyrrolidinyl group, piperidyl group, piperidino group, morpholinyl group, morpholino group, piperazinyl group, pyrrolyl group, imidazolyl group, or pyridyl group. The alkyl moiety (alkylene group) is, but not limited to, preferably an alkylene group having 1 to 6 carbon atoms, and more preferably an alkylene group having 1 to 3 carbon atoms. Specifically, the examples of a heterocycle group include a piperidinomethyl group, a pyrrolidinylmethyl group, a piperidylmethyl group, a morpholinylmethyl group, a morpholinomethyl group, a piperazinylmethyl group, a pyrrolylmethyl group, an imidazolylmethyl group, a pyridylmethyl group, a (1-pyrrolidinyl)butyl group, a morpholinopropyl group, a 1,1 dimethyl-2-(1-pyrrolidinyl)ethyl group, a 1,1-dimethyl-2-piperidinoethyl group, a 1,1-dimethyl-3-(imidazol-1-yl)propyl group, a (2,6-dimethylpiperidino)methyl group, a (2,6-dimethylpiperidino)ethyl group, a (2,6-dimethylpiperidino)propyl group, etc.

A 5 to 7 membered heterocyclic ring, which may have a substituent, is preferably a 5- or 6-membered heterocyclic ring, most preferably a 6-membered heterocyclic ring. The heterocyclic ring is, but not limited to, a saturated or unsaturated heterocyclic ring, preferably a saturated heterocyclic ring. The examples of 5- to 7-membered heterocyclic ring are morpholine (tetrahydro-1,4-oxazine), piperazine, pyrrolidine, imidazolidine, pyrazolidine, piperidine, imidazole, pyridine, tetrahydro-1,3-oxazine, or tetrahydro-1,2-oxazine.

The examples of the substituent include a hydroxyl group, an alkyl group having 1 to 6 carbon atoms, an alkoxy group having 1 to 6 carbon atoms, or a phenylalkyl group having 1 to 6 carbon atoms. A phenylalkyl group having 6 to 12 carbon atoms is an alkylene group bonded to a phenyl group. An alkyl moiety (alkylene group) is, but not limited to, preferably an alkylene group having 1 to 6 carbon atoms, and more preferably an alkylene group having 1 to 3 carbon atoms.

Aldehyde group is a group represented by—CHO. A hydroxy group is a group represented by—OH.

An alkyl ester group having 1 to 6 carbon atoms is a group in which an alkyl group having 1 to 6 carbon atoms is bonded to an ester group (—COO—). An alkyl group having 1 to 6 carbon atoms is as described above.

An alkyl ester alkylene group is a group in which an alkyl group having 1 to 6 carbon atoms is bonded to an ester group (—COO—) and further bonded to alkylene group having 1 to 6 carbon atoms. An alkyl group having 1 to 6 carbon atoms is as described above. The examples of an alkylene group having 1 to 6 carbon atoms include a methylene group, an ethylene group, a n-propylene group, an isopropylene group, a n-butylene group, an isobutylene group, a t-butylene group, a n-pentylene group, a 2-methylbutylene group, a 2,2-dimethylpropylene group, a n-hexylene group, or a heptylene group.

Compound A is a derivative of 4-phenylcoumarin. 4-Phenylcoumarin and a derivative thereof can be synthesized by various methods. Compound B is a derivative of indole or indoline. Indole or indoline and derivatives thereof can also be synthesized by various methods. For example, indole can be synthesized by a gas phase reaction of aniline and ethylene glycol in the presence of catalyst, using aniline as the starting material. These compounds can also be purchased commercially.

The examples of compound A include, but not limited to, compound 10-0, compound 10-1, compound 10-2, compound 10-3, compound 10-4, compound 10-5, and C-30 represented by the following formulae:

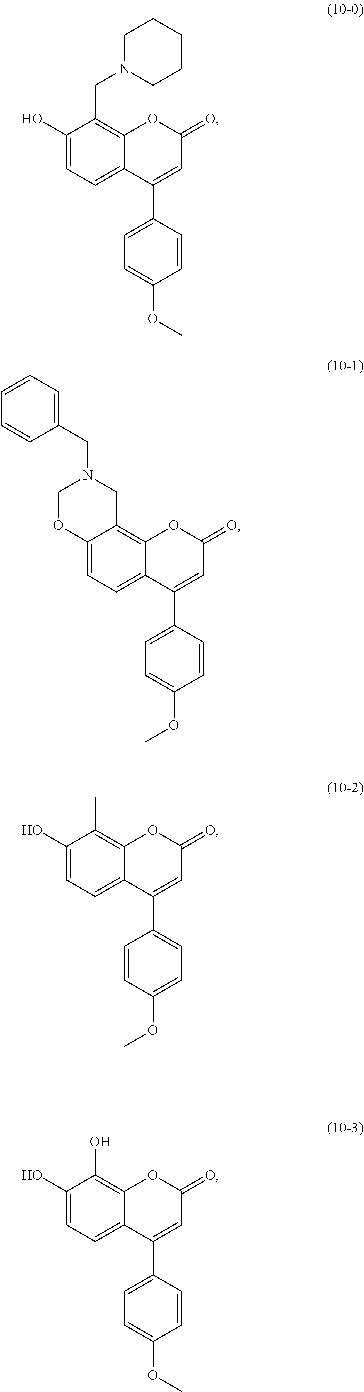 [SMILES: COc1ccc(-c2cc(=O)oc3c(CN4CCCCC4)c(O)ccc23)cc1 ; COc1ccc(-c2cc(=O)oc3c4c(ccc23)OCN(Cc2ccccc2)C4)cc1 ; COc1ccc(-c2cc(=O)oc3c(C)c(O)ccc23)cc1 ; COc1ccc(-c2cc(=O)oc3c(O)c(O)ccc23)cc1]

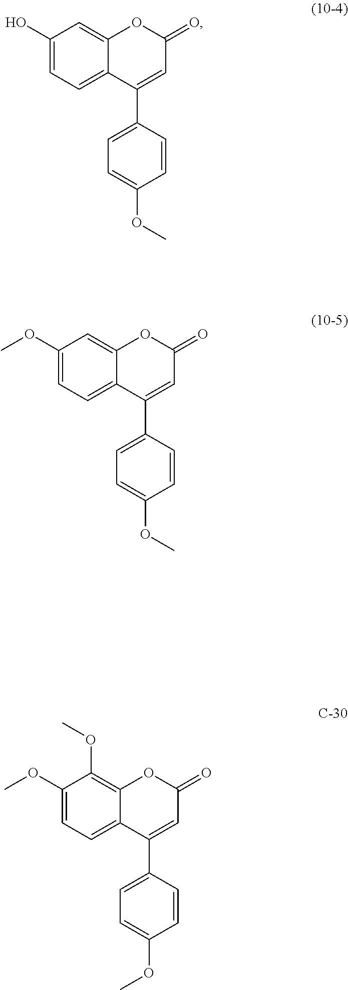 [SMILES: COc1ccc(-c2cc(=O)oc3cc(O)ccc23)cc1 ; COc1ccc(-c2cc(=O)oc3cc(OC)ccc23)cc1 ; COc1ccc(-c2cc(=O)oc3c(OC)c(OC)ccc23)cc1]

The examples of compound B include, but not limited to, compounds 14-0, 14-1, 14-2, 14-3, 14-4, 14-5, 14-6, and 14-7 represented by the following formulae:

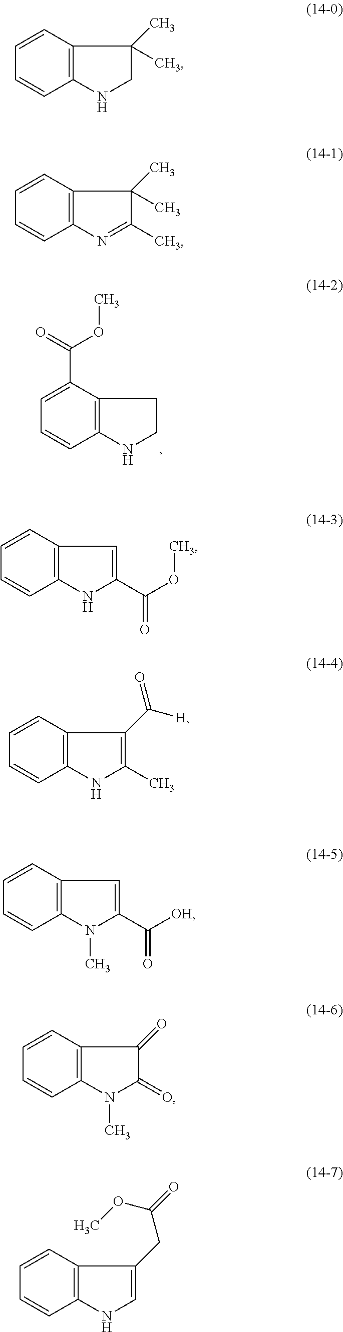

The salts of compound A, compound B, and compound C are pharmaceutically acceptable salts, which may be acid addition salts or salts with bases, depending on the type of substituent. Specifically, the examples of acid addition salts or salts with bases include acid addition salts with inorganic acids such as hydrochloric acid, hydrobromic acid, hydroiodic acid, sulfuric acid, nitric acid, and phosphoric acid, and organic acids such as formic acid, acetic acid, propionic acid, oxalic acid, malonic acid, succinic acid, fumaric acid, maleic acid, lactic acid, malic acid, mandelic acid, tartaric acid, dibenzoyl tartrate, ditoloyl tartrate, citric acid, methanesulfonic acid, ethanesulfonic acid, benzenesulfonic acid, p-toluenesulfonic acid, aspartic acid, and glutamic acid, salts with inorganic bases such as sodium, potassium, magnesium, calcium, and aluminum, and organic bases such as methylamine, ethylamine, ethanolamine, lysine, and ornithine, salts with various amino acids and amino acid derivatives such as acetylleucine, and ammonium salts, etc.

Furthermore, the active ingredients used in the present invention also include various hydrates, solvates, and crystalline polymorphs of compound A, compound B, and compound C and salts thereof. The present invention also includes compounds labeled with various radioactive or non-radioactive isotopes.

Prostate cancer, which is the therapeutic target of the pharmaceutical composition of the present invention, is cancer that develops in the prostate gland and an antiandrogenic agent may be administered as hormone therapy. The type of prostate cancer to be treated is, but not limited to, androgen-independent prostate cancer (CRPC) that has acquired resistance to hormone therapy, which is a particularly effective target. As shown in the Examples described later, the pharmaceutical composition of the present invention can be effectively used for CRPC that has acquired resistance to hormone therapy, since the pharmaceutical composition shows remarkable anticancer effects on AR-positive CRPC and AR-negative CRPC. However, the pharmaceutical composition can also be used to treat prostate cancer that is not hormone therapy-resistant or CRPC by other mechanisms.

Breast cancer, which is the therapeutic target of the pharmaceutical composition of the present invention, is a carcinoma that develops in the breast tissue, and an anti-estrogen agent may be administered as hormone therapy. The type of breast cancer to be treated is, but not limited to, estrogen-independent breast cancer that has developed resistance to hormone therapy, which is a particularly effective target. However, the pharmaceutical composition can also be used to treat breast cancer that is not hormone therapy-resistant or resistant to treatment by other mechanisms.

The inventors of the present invention have found that RNA-binding protein PSF (PTB-associated Splicing Factor) regulates factors involved in the malignant transformation of cancer in prostate cancer or breast cancer that has acquired resistance to hormone therapy. For example, PSF upregulates the expression of AR or its spliced variant, AR-V7, which is increased in Castration-resistant prostate cancer (CRPC). PSF expression is also upregulated in breast cancer OHTR cells that have acquired resistance to hormone therapy compared with the parental MCF7 cells that is sensitive to hormone therapy.

The compound A, compound B, and compound C used in the present invention can bind to PSF. As shown in the Examples, the expression of AR, AR-V7, and SchLaP1, which are target genes of PSF at the RNA level, can be inhibited in 22Rv1 cells, a model cell for hormone-resistant prostate cancer. In addition, in OHT-TamR cells, a hormone-resistant model of breast cancer, the expression of ERa and SCFD2, which are downstream genes of PSF, can be inhibited.

That is to say, it is considered that compound A, compound B, and compound C are effective for the treatment of hormone therapy-resistant prostate cancer or breast cancer by binding to PSF and suppressing the function of PSF.

A pharmaceutical composition containing one or more of the above-mentioned compound A, compound B, or compound C, or salts thereof as an active ingredient can be prepared by using excipients commonly used in the art, that is to say, excipients for pharmaceutical compositions and drug carriers, etc., by a method commonly used in the art.

Administration may be oral in pill, capsule, granule, powder, or liquid form, etc., or parenteral in injection such as intra-articular, intravenous, or intramuscular, suppository, eye drop, eye ointment, transdermal solution, ointment, transdermal patch, transmucosal solution, transmucosal patch, inhalation, etc.

The examples of solid composition for oral administration include tablets, dispersions, and granules. In such solid compositions, one or more active ingredients are mixed with at least one inert excipient, such as lactose, mannitol, glucose, hydroxypropyl cellulose, microcrystalline cellulose, starch, polyvinylpyrrolidone, and/or magnesium metasilicate. The composition may contain inert additives, such as lubricants e.g., magnesium stearate, disintegrants e.g., sodium carboxymethylstatinate, stabilizer, solubilizer, etc., according to conventional methods. Tablets or pills may be coated with a sugar coating or a film of a gastric or enteric soluble substance as needed. The examples of liquid composition for oral administration include pharmaceutically acceptable emulsion, solution, suspension, syrup, or elixir, which contain commonly used inert diluent, such as purified water or ethanol. In addition to the inert diluent, the liquid composition may contain auxiliary agents such as solubilizer, wetting agent, and suspending agent, sweetening agent, flavoring agent, aromatic agent, and preservative.

The examples of injectable drug for parenteral administration include sterile aqueous or non-aqueous solution, suspension or emulsion. The examples of aqueous solvent include, distilled water or saline solution for injection. The examples of nonaqueous solvent include, propylene glycol, polyethylene glycol or vegetable oils such as olive oil, alcohols such as ethanol, or polysorbate 80 (pharmacopeia name). Such composition may further contain isotonicity agent, preservative, wetting agent, emulsifier, dispersant, stabilizer, or solubilizer. These are sterilized, for example, by filtration through a bacteria-retaining filter, the addition of a bactericidal agent, or irradiation. These can also be produced as sterile solid composition and dissolved or suspended in sterile water or sterile injectable solvent, prior to use.

The examples of topical products include ointments, hard plasters, creams, jellies, poultices, sprays, lotions, eye drops, eye ointments, etc. The examples of topical products also include commonly used ointment bases, lotion bases, aqueous or non-aqueous liquids, suspensions, emulsions, etc. The examples of ointment or lotion bases include polyethylene glycol, propylene glycol, white vaseline, white beeswax, polyoxyethylene hardened castor oil, glycerin monostearate, stearyl alcohol, cetyl alcohol, lauromacrogol, sesquioleinic acid sorbitan, etc.

Transmucosal agents, such as inhalants and nasal spray, may be used in solid, liquid, or semi-solid form and can be manufactured according to conventionally known methods. For example, known excipients, pH adjusters, preservatives, surfactants, lubricants, stabilizers and thickeners may be added appropriately. Any suitable devices for inhalation or air delivery may be used for administration. For example, transmucosal agents may be administered using known devices such as metered dose inhalation devices or atomizers, as a compound alone or as a powder in a formulated mixture, or as a solution or suspension in combination with a pharmaceutically acceptable carrier. Dry powder inhalers and the like may be used for single or multiple doses, and dry powder or powder-containing capsules may be used. Alternatively, transmucosal agents may be administered in the form of a pressurized aerosol spray using suitable ejection agents, such as preferred gases, for example, chlorofluoroalkane, hydrofluoroalkane, or carbon dioxide, etc.

Dosage varies depending on the type of disease, symptom, age, and gender of patients. The usual dosage for oral administration is approximately 0.001 mg/kg-500 mg/kg per day for adults, which is administered once or divided into two to four doses. When administered by injection, injection is administered as a rapid intravenous infusion or intravenous drip at a dose of approximately 0.0001 mg/kg-10 mg/kg once or twice per day for adults. When administered by inhalation, a single or multiple doses of approximately 0.0001 mg/kg-10 mg/kg per adult per day is administered. When administered by transdermal drug, transdermal drug is applied once or twice per day at a dose of approximately 0.01 mg/kg-10 mg/kg per day for adults.

Compound A, compound B, or compound C, or salts thereof, may be used in combination with various therapeutic or prophylactic agents for diseases for which compound A or compound B, or salts thereof is considered to be effective. The combination use may be simultaneous, or separate and consecutive, or at desired time intervals. The simultaneous administration formulation may be a compounding agent or a separate formulation.

### <<Method for Treating Prostate Cancer or Breast Cancer>>

Compound A, compound B, or compound C may be used for the treatment of prostate cancer or breast cancer. That is to say, this specification discloses a method for treating prostate cancer or breast cancer comprising the step of administering a therapeutically effective amount of a compound represented by the formula (1) or a salt thereof, or a compound represented by the formula (2) or (3) or a salt thereof, or compound C or a salt thereof, to a patient having prostate cancer or breast cancer patient.

### <<Compound a, Compound B, or Compound C for Use in the Treatment of Prostate or Breast Cancer>>

Compound A, compound B, or compound C may be used for the treatment of prostate cancer or breast cancer. That is to say, this specification discloses a compound represented by the formula (1) or a salt thereof, or a compound represented by the formula (2) or (3) or a salt thereof, or compound C or a salt thereof for use in the treatment of prostate or breast cancer.

### <<Use of Compound a, Compound B, or Compound C in the Manufacture of Pharmaceutical Composition>>

Compound A, compound B, or compound C may be used for the manufacture of a pharmaceutical composition for the treatment of prostate cancer or breast cancer.

That is to say, this specification discloses use of a compound represented by the formula (1) or a salt thereof, or a compound represented by the formula (2) or (3) or a salt thereof, or compound C or a salt thereof in the manufacture of a pharmaceutical composition for the treatment of prostate cancer or breast cancer.

### <<Function>>

The mechanism by which compound A or compound B is effective in the treatment of prostate cancer or breast cancer has not been analyzed in detail, but can be estimated as follows. A compound A is a derivative of 4-phenylcoumarin. The skeleton of 4-phenylcoumarin is considered to be important for binding to PSF (anticancer effect). Furthermore, the presence of an oxygen atom at the 7-position of coumarin is considered to enhance the anticancer effect. A compound B is a derivative of indole or indoline. The skeleton of indole or indoline is considered to be important for the binding to PSF (anticancer effect).

### EXAMPLES

The present invention will now be further illustrated by, but is by no means limited to, the following Examples.

### Example 1

In this Example, the effects of compound A and compound B on the proliferative ability of 22Rv1, a model cell line of hormone therapy-resistant prostate cancer, and LTAD (long time androgen deprived) cells, a model of hormone therapy resistance, were examined.

Cells were seeded in a 96-well plate (3×10³ cells/well), and compound 10-0 and compound 14-0 were added at the concentration of 30 μM. After 3 days, cell proliferative ability was evaluated by MTS assay. The MTS assay was performed as follows:

The reaction was carried out using Cell titer 96 (Promega). The MTS assay is an assay to measure the number of viable cells that based on the reduction reaction of tetrazolium salt [MTS;3-(4,5-dimethylthiazol-2-yl)-5-(3-carboxymethoxyphenyl)-2-(4-sulfophenyl)-2H-tetrazolium, inner salt] to the formazan product that is a chromogenic substance. After 1 hour incubation, the absorbance is measured using a microplate reader at 490 nm to evaluate cell proliferative ability.

Furthermore, the MST assay was performed in the same manner using LTAD and VCaP cells by adding 1 μM or 10 UM of compound 10-0, 10-1, 10-3, 14-0, or 14-5.

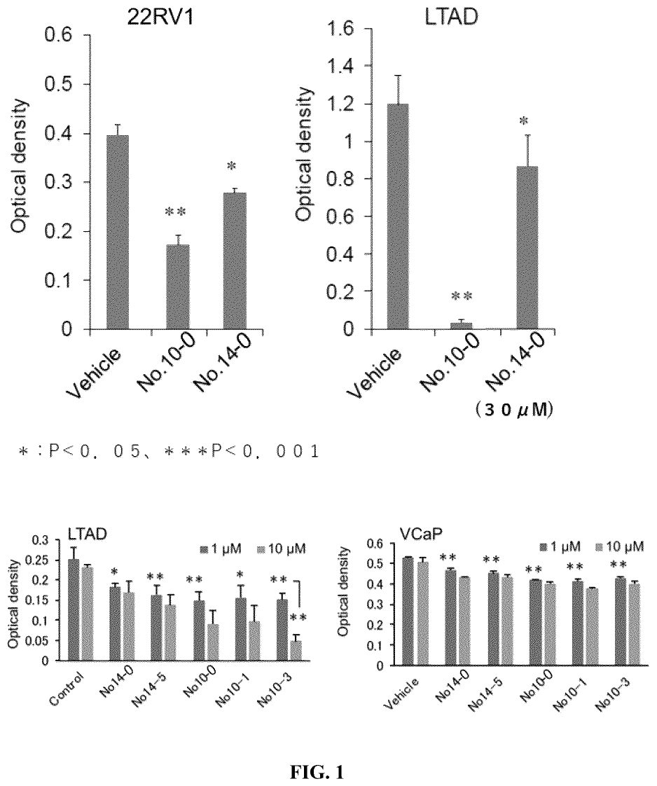

The results showed that all the compounds 10-0, 10-1, 10-3, 14-0, and 14-5 significantly suppressed cell proliferation (FIG. 1).

### Example 2

In this Example, the inhibitory ability of compounds 10-0, 10-1, 10-2, 10-3, 10-4, 14-0, and 14-5 to the interaction of PSF with its target RNA was examined by RNA pull-down method.

A biotin-labeled probe for CTBP1-AS was prepared using Biotin RNA Labeling Mix (Roche). The RNA probe was prepared by T7 RNA polymerase using a DNA strand in which 1.5 kb of CTBP1-AS was incorporated into T7 promoter as a template. RNA was mixed with nuclear extracts extracted from cells with RIP buffer and rotated at 4° C. for 8 hours for binding. Avidin beads were added and the mixture was rotated for 2 hours to collect biotinylated RNA probes. The beads were collected by centrifugation, washed with buffer three times, and then SDS sample buffer was added to the beads, and the bound proteins were examined by Western blotting. 22Rv1 cells were used to prepare nuclear extracts in the same manner as in Example 1, except the concentrations of the added compounds were varied.

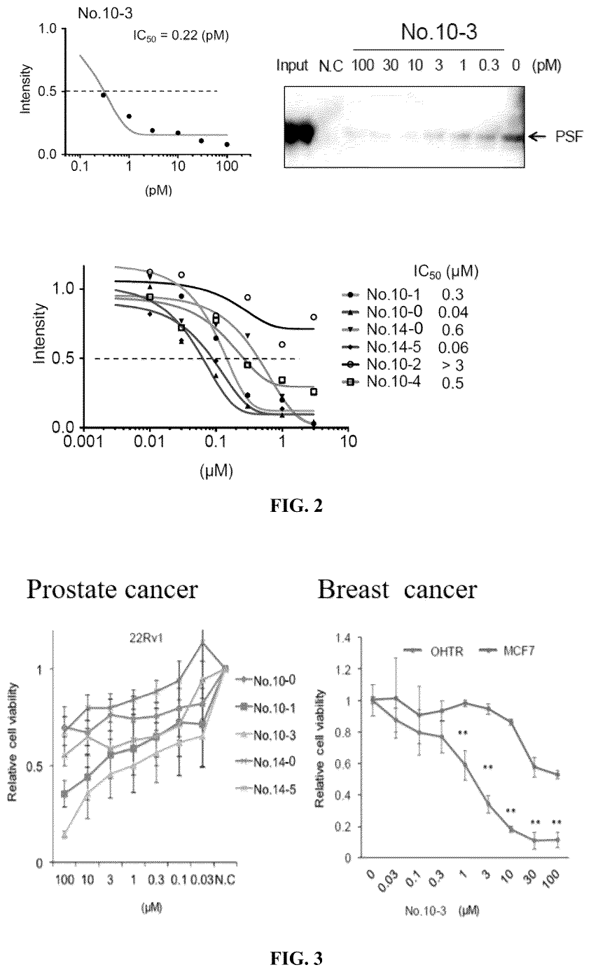

As shown in FIG. 2, all compounds inhibited the binding of PSF to RNA.However, the inhibitory effect of compound 10-3 was particularly strong (IC50=0.22 μM).

### Example 3

In this Example, the effects of compounds 10-0, 10-1, 10-3, 14-0, and 14-5 on cell proliferation were examined using a model cell line of hormone therapy-resistant prostate cancer 22Rv1, breast cancer cell MCF7, and a model of hormone therapy-resistant OHTR cells established from MCF7 cells. The MTS assay was performed as in Example 1.

As shown in FIG. 3, each compound inhibited 22Rv1 cell proliferation. Compound 10-3 showed stronger inhibitory effect on cell proliferation of hormone therapy-resistant OHTR cells than normal breast cancer cells, MCF7.

### Example 4

In this Example, the expression levels of PSF target genes at the RNA level, AR, AR-V7, SchLaP1, and downstream signals of AR, FKBP5 and ACSL3, were examined by adding compounds 10-0, 10-1, and 10-3 to 22Rv1 cells.

22Rv1 cells were treated with compounds 10-0, 10-1, and 10-3. After 48 hours incubation, total RNA was collected to examine changes in mRNA expression of the target genes. This analysis was performed by quantitative real-time PCR as follows

Total RNA was collected from cells using ISOGEN (NIPPON Gene). cDNA was synthesized using Prime script RT reagent kit (TaKaRa Bio). Step one real-time PCR (Applied biosystem) and KAPA SYBR Fast PCR kit (NIPPON Genetics) were used to measure the mRNA expression levels of each gene and the internal control, GAPDH. The expression levels for GAPDH were calculated from the number of cycles using the ΔΔCt method.

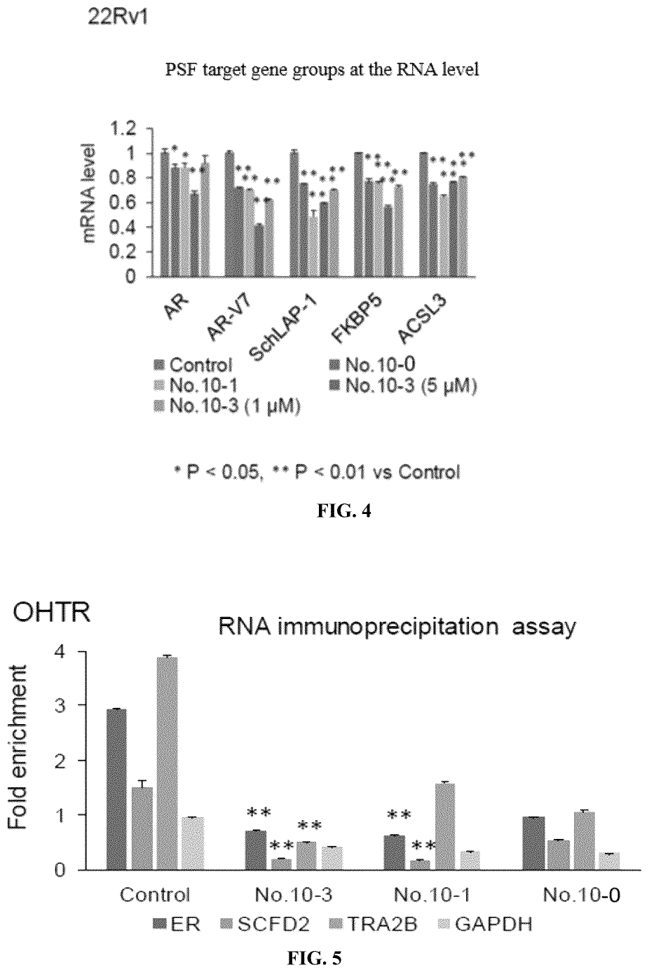

It was confirmed that the mRNA expression of AR, AR-V7, SchLaP1, FKBP5, and ACSL3 was suppressed by the administration of compound 10, 10-1, or 10-3 (10 μM) (FIG. 4).

### Example 5

In this Example, compounds 10-0, 10-1, and 10-3 were added to hormone therapy resistant breast cancer model OHT-TamR cells. After 48 hours of cultivation, the cells were collected, lysed, and immunoprecipitated with PSF antibody. Then total RNA binding to PSF protein was extracted. Quantitative PCR was used to quantify the amount of RNA bound to PSF for ERα, SCFD2, and TRA2B, which are target genes of PSF at the RNA level. As a control, GAPDH-bound RNA was also quantified.

OHT-TamR cells were treated with compounds 10-0, 10-1, and 10-3 for 48 hours. Total RNA binding to PSF protein was collected from immunoprecipitated beads, and the amount of each target gene and GAPDH as a control was measured. Quantitative real-time PCR was performed as in Example 4.

It was confirmed that the binding of ERα, SCFD2, and TRA2B to PSF protein was significantly inhibited by the administration of compound 10-3 (10 μM) compared to GAPDH, and binding of ERa and SCFD2 was significantly inhibited by the administration of 10-1 (FIG. 5).

### Example 6

In this Example, anticancer effect of compound 10-3 in vivo was examined using a xenograft mouse model.

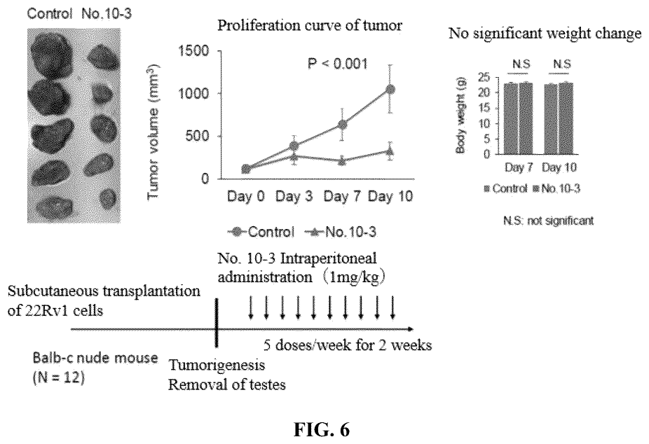

For tumor transplantation, 22Rv1 cells were adjusted to 1×10⁷, mixed with Matrigel at 1:1, and injected subcutaneously into 6-week-old male nude mice (BALB/cAJcl-nu/nu). Five to eight days after subcutaneous injection, testes were removed when the tumor volume reached about 100-200 mm³, to prepare a model of hormone therapy refractory CRPC tumors. Six mice were injected intraperitoneally with compound 10-3 at a concentration of 1 mg/kg. Vehicle (DMSO) was administered as a control. The treatment was repeated 5 times/week for 2 weeks, and the tumor diameter was measured (FIG. 6).

As shown in FIG. 6, tumor growth was significantly suppressed in mice treated with compound 10-3. On the other hand, administration of No. 10-3 did not significantly reduce the body weight of the mice.

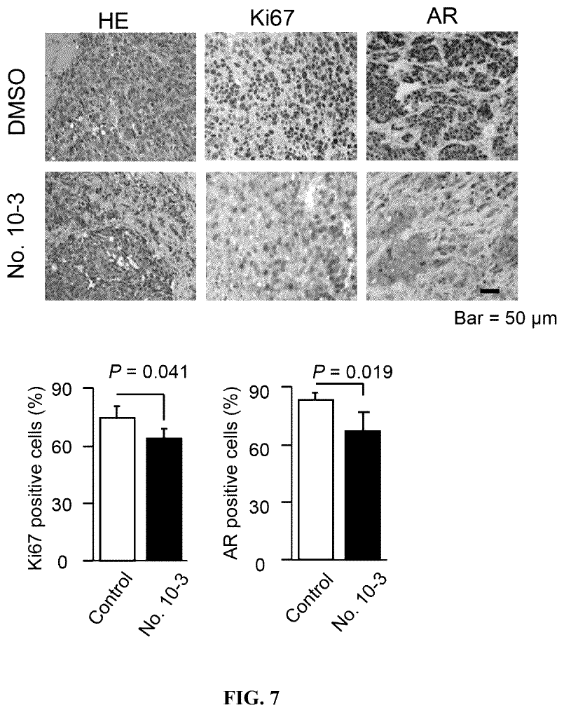

In addition, administration of compound 10-3 reduced the number of cells expressing Ki67 and AR in tumor tissues (FIG. 7).

### Example 7

In this Example, the effects of compounds 10-1 and 10-3 on cell proliferation were examined using an AR-negative CRPC model DU145 cells. The MTS assay was performed as in Example 1.

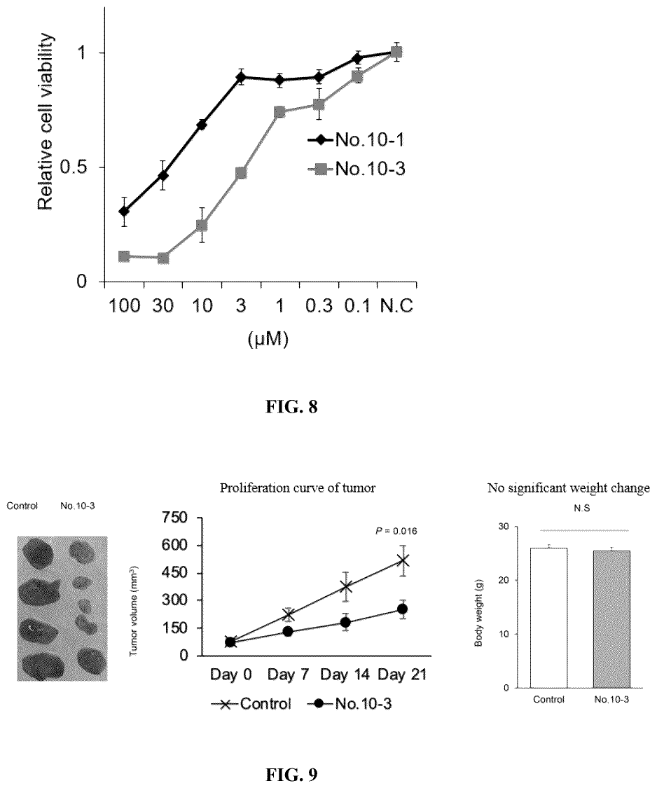

As shown in FIG. 8, each compound inhibited cell proliferation in DU145 cells.

### Example 8

In this Example, anticancer effect of compound 10-3 in vivo was examined using an AR-negative CRPC tumor xenograft model, in which DU145 cells were subcutaneously transplanted to mice.

For tumor transplantation, DU145 cells were adjusted to 1×10⁷, mixed with Matrigel at 1:1, and injected subcutaneously into 6-week-old male nude mice (BALB/cAJcl-nu/nu). Five to eight weeks after subcutaneous injection, when the tumor volume reached about 100-200 mm³, compound 10-3 was injected intraperitoneally at a concentration of 5 mg/kg into 8 mice (the photo shows 4-5 mice). Controls were injected with Vehicle (DMSO). The treatment was repeated 5 times/week for 3 weeks, and the tumor diameter was measured (FIG. 8).

As shown in FIG. 8, tumor growth was significantly inhibited in mice treated with compound 10-3. On the other hand, administration of No. 10-3 did not significantly reduce the body weight of the mice. The compounds of the present invention can inhibit the expression of AR in cancer cells, but they also show anticancer activity against AR-negative cancer cells. Therefore, it is considered that the compound exerts its anticancer effect through pathways other than suppression of AR expression.

### Example 9

In this Example, the binding site of compound 10-3 to PSF, which is the target protein of the present invention, was examined by docking assay. Furthermore, mutations were introduced into the amino acids of PSF that are predicted to interact with compound 10-3, and the inhibitory effect of compound 10-3 on the interaction of PSF with its target RNA was examined.

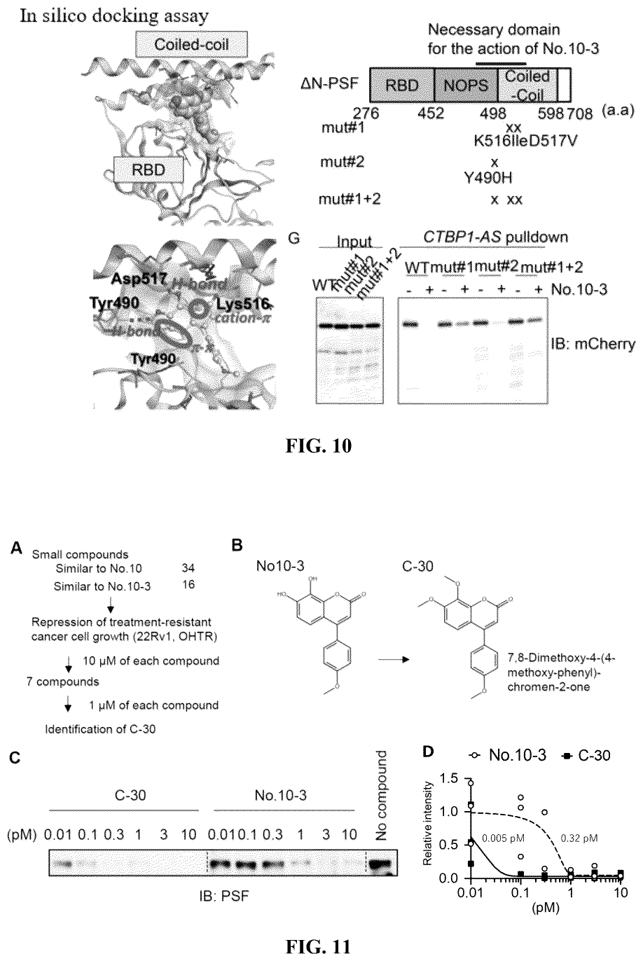

The crystal structure of human PSF (276-535 amino acids) was downloaded from the Protein Data Bank (PDB). The volume and shape of the binding cavities were analyzed using the SiteFinder module of the Molecular Operation Environment (MOE) program. As a result, compound 10-3 was estimated to bind to the Coild-Coil site from the NOPS of PSF, as shown in FIG. 10.

The following combinations of mutations were introduced into the above-mentioned presumed site at position 490, tyrosine (Y), position 516, lysine (K), and position 517, aspartic acid (D).
mut #1: K516I, D517V
mut #2: Y490H
mut #1+2: Y490H, K516I, D517V

The binding of mut #1, mut #2, mut #1+2, or wild-type PSF to the RNA strand of CTBP1-AS was examined by RNA pull-down method. The inhibitory effect for binding of compound 10-3 was measured by adding compound 10-3. Compared to the wild type, mut #1, mut #2, or mut #1+2s mutant showed the decreased inhibitory effect on the PSF binding to the RNA probe by the addition of compound 10-3. Therefore, it is likely that compound 10-3 binds to tyrosine (Y) at position 490, lysine (K) at position 516, and aspartate (D) at position 517 in the region spanning from NOPS to Coild-Coil of PSF.

### INDUSTRIAL APPLICABILITY

The pharmaceutical composition of the present invention can be effectively used for the treatment of prostate cancer and breast cancer.

### Example 10

In this example, we identified a small compound with improved PSF inhibition activity and examined anticancer activity of the compound (C-30) by using an MTS assay and a p53-mutated cancer cell xenograft model.

### (1) Materials and Methods

### Cell Culture

The cell lines used in this study were obtained from the American Type Culture Collection (ATCC). OHTR cells were established at Saitama Medical University (Mitobe Y, Iino K, Takayama K, Ikeda K, Suzuki T, Aogi K, et al. PSF Promotes ER-Positive Breast Cancer Progression via Posttranscriptional Regulation of ESR1 and SCFD2. Cancer Res 2020; 80:2230-2242.). MDA-MB-231 (RRID:CVCL_0062) and OHTR cells were cultured in DMEM supplemented with 10% fetal bovine serum (FBS), 50 U/mL penicillin, and 50 μg/mL streptomycin. DU145 (RRID:CVCL_0105), PC3 (RRID:CVCL_0035), and 22Rv1 (RRID:CVCL_1045) cells were cultured in RPMI medium supplemented with 10% fetal bovine serum (FBS), 50 U/mL penicillin, and 50 μg/mL streptomycin. Cell identity was validated by short tandem repeat (STR) analysis in 2019 (BEX Co. Ltd., Tokyo, Japan), and cell stocks were used for further experiments. All the cell lines were grown at 37° C. in a 5% CO₂ atmosphere. The cells were routinely checked for mycoplasma contamination using a PCR-based Mycoplasma Detection Kit (Jena Bioscience, Thuringia, Germany). Additional viral infections were examined by PCR (ICR Monitoring Center, Kanagawa, Japan). Stocks of low-passage cells were maintained for the experiments, and cell cultures were subcultured in fresh vials at least once per month.

### In Vitro Transcription and RNA Labeling

A linear template of CTBP1-AS was prepared by treating pcDNA3.0-CTBP1-AS with EcoRV and precipitating it with ethanol. As previously described (Takayama K, Matsuoka S, Adachi S, Honma T, Yoshida M, Doi T, et al. Identification of small-molecule inhibitors against the interaction of RNA-binding protein PSF and its target RNA for cancer treatment. PNAS Nexus 2023; 2 (6): pgad203.), the biotin-labeled CTBP1-AS probe was produced using Biotin RNA Labeling Mix (Roche, Basel, Switzerland) and T7 RNA polymerase. Biotinylated RNAs were treated with RNase-free DNase (Qiagen), and 10 μmol of biotinylated RNA was heated to 60° C. for 10 minutes and slowly cooled to 4° C. before use in the RNA pull-down assay.

### RNA Pulldown Assay

The biotin-labeled RNA probe of CTBP1-AS was mixed with 100 μg of pre-cleared nuclear extracts in RIP buffer (150 mM KCl, 25 mM Tris pH 7.4, 5 mM EDTA, 0.5 mM DTT, 0.5% NP40) supplemented with yeast tRNA (10 μg/mL) (Thermo Fisher, Waltham, MA, USA) and rotated at 4° C. for eight hours. Washed streptavidin agarose beads (30 μL) (Thermo Fisher) were added and incubated for one hour at 4° C. The beads were washed three times with RIP buffer and boiled in SDS buffer. The retrieved proteins were analyzed by western blotting. Small molecules were added to the nuclear lysates five hours before mixing with RNA probes to analyze their effects.

### MTS Assay

The cells (1×10³) were plated and cultured in 96-well plates. After three days of incubation, a [3-(4,5-dimethylthiazol-2-yl)-5-(3-carboxymethoxyphenyl)-2-(4-sulfophenyl)-2H-tetrazolium, inner salt] (MTS) assay was performed using the CellTiter 96 Aqueous Kit (Promega, Madison, WI) according to the manufacturer's instructions. Background correction used a blank well containing no reagents.

### Xenograft Model

MDA-MB231 and DU145 cells (3×10⁶) were mixed with Matrigel (BD Biosciences) and subcutaneously injected into BALB/c nude mice. No. 10-3, C-30, or the control (vehicle) was injected intraperitoneally three times per week for three to four weeks.

The number of mice and the amounts of small compounds injected are described in Figure Legends. The small compounds were dissolved in PBS containing 2% DMSO/10% Tween80 (Nakarai). For the 22Rv1 xenograft experiments, castration was performed after tumor development. Tumor size was measured using calipers. Tumor volume was calculated using the formula V=0.5×r1×r2×r3 (r1> r2>r3). To evaluate toxicity, the mice's body weights were recorded weekly (Takayama K, Suzuki T, Fujimura T, Yamada Y, Takahashi S, Homma Y, et al. Dysregulation of spliceosome gene expression in advanced prostate cancer by RNA-binding protein PSF. Proc Natl Acad Sci USA 2017; 114:10461-10466; Takayama K, Honma T, Suzuki T, Kondoh Y, Osada H, Suzuki Y, et al. Targeting Epigenetic and Posttranscriptional Gene Regulation by PSF Impairs Hormone Therapy-Refractory Cancer Growth. Cancer Res 2021; 81:3495-3508.). Basic principles of animal experiments (replacement, reduction, and refinement) were considered in this study. This study was approved by the Institutional Ethical Committee of the Tokyo Metropolitan Geriatric Hospital and Institute of Gerontology (#0021). After the mice were euthanized, blood samples were obtained from their hearts for serum biochemical analysis (Oriental Kobo, Shiga, Japan). The tumors were homogenized in NP40 buffer containing a protease inhibitor cocktail (Nacalai, Tokyo, Japan) for western blot analysis. For immunohistochemistry (IHC), tumors were formalin-fixed and embedded in paraffin.

Statistics and reproducibility

All experiments were performed at least twice, and similar results were obtained. Data are expressed as the mean±S.D. Two-sided Student's t-tests were performed to determine statistical significance between the two groups. When more than two samples were compared, a one-way analysis of variance (ANOVA) with a post hoc Tukey test was performed. The statistical tests are described in the figure legends. Statistical significance was defined as P<0.05. The qPCR analysis of the cell lines was performed using technical replicates. Biological replicates were used for all the other analyses. MS Excel (Microsoft Corp., Redmond, WA, USA) and GraphPad Prism ver. 6.0 (La Jolla, CA, USA) were used for statistical analyses.

### (2) Results

Identification of a Small Compound with Improved PSF Inhibition Activity

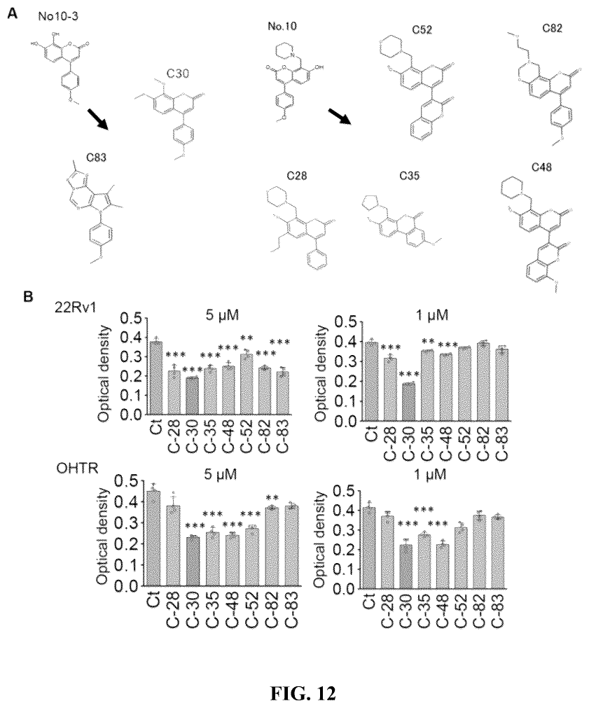

To identify more effective PSF-specific inhibitors as potential drugs against treatment-resistant cancer cells, we screened potential inhibitors based on similarity to No. 10-3 (FIGS. 11A and 12A). Fifty candidate molecules were identified, and their effects on cancer cell proliferation were analyzed. We used the 4-hydroxytamoxifen (OHT)-resistant (OHTR) cell line established from MCF7 cells as a hormone-refractory breast cancer model, in addition to the anti-androgen therapy-resistant 22Rv1 prostate cancer cell line. We found that one of the identified compounds, C-30 (FIG. 11B), significantly decreased the viability of both cell lines at a low dose (1 μM) (FIG. 12B). Next, we assessed the effect of C-30 on the interaction between PSF and CTBP1-AS RNA probes (FIG. 11C). We compared the inhibitory activity of C-30 with that of No. 10-3, showing that the IC50 of C-30 for inhibition (0.005 μM) was lower than that of No. 10-3 (0.32 μM) (FIGS. 11C and 11D).

### C-30 Treatment Represses Hormone-Receptor-Positive Cancer Cell Growth

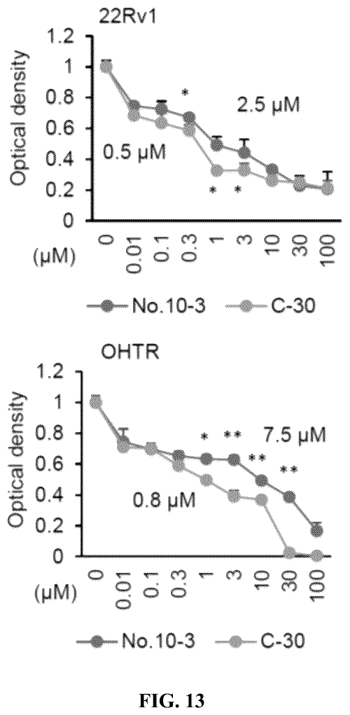

An MTS assay confirmed that C-30 inhibited PSF activity; its effect on cell growth was compared with that of No. 10-3. The repression of prostate cancer cell proliferation by C-30 was more evident in a CRPC model 22Rv1 cells (IC50=0.5 μM) compared with No. 10-3 (IC50=2.5 μM). We also observed that C-30 treatment inhibited the proliferation of OHTR breast cancer cells more evidently compared with No. 10-3 (FIG. 13).

Enhanced Effect of C-30 on Cancer Cells Expressing p53-Mutant by Reactivation of p53 Signaling

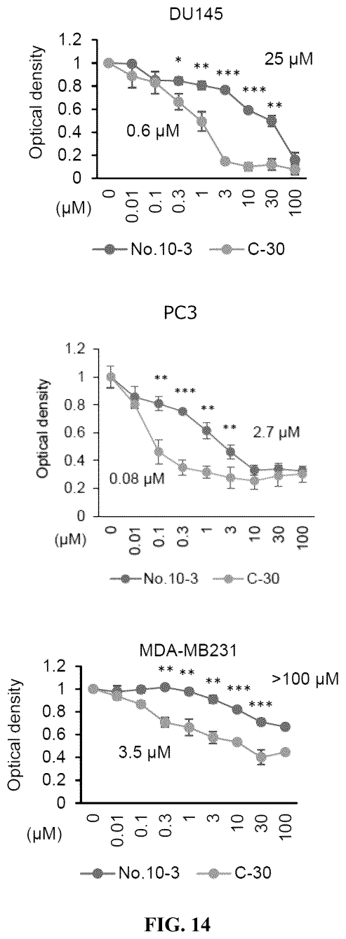

An MTS assay used p53 mutated and hormone receptor-negative cells. Surprisingly, C-30 treatment blocked DU145, PC3, and MDA-MB231 cell growth, with IC50 levels of 0.6 μM, 0.08 UM and 3.5 M, respectively. (FIG. 14).

C-30 Represses Tumor Growth in p53-Mutated Cancer Cell Xenograft Model

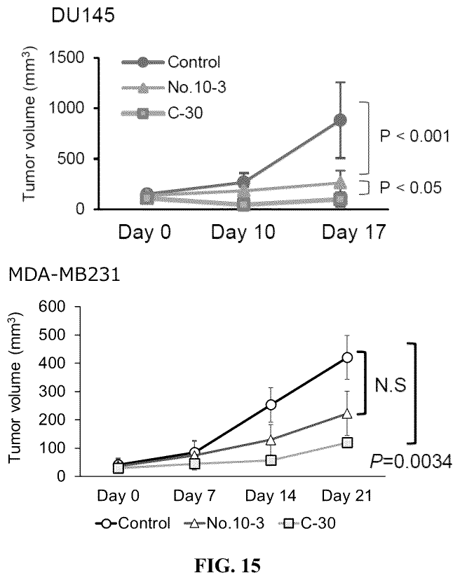

We investigated the ability of C-30 to inhibit PSF activity in an in vivo xenograft model of hormone receptor-negative cells expressing mutated p53. In the DU145 xenograft model, tumor growth was significantly inhibited by No. 10-3 treatment (10 mg/kg, three times per week); however, an even higher level of inhibition was observed upon C-30 treatment (FIG. 15).

We further tested the efficacy of C-30 and No. 10-3 treatments in inhibiting tumor growth in xenograft models of ERα-negative and p53-mutated breast cancer MDA-MB231 cells (FIG. 16). We observed significant tumor growth inhibition after administering C-30 (15 mg/kg, five times per week), with no apparent toxic effects in mice. In contrast, the same amount of No. 10-3 injection was not as effective.

1. A method for treating prostate cancer comprising: a step of administering a therapeutically effective amount of a compound represented by a following formula (1): 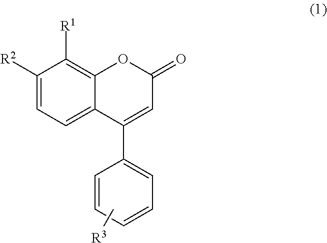
wherein R¹ is a hydrogen atom, a hydroxyl group, an alkyl group having 1 to 6 carbon atoms, an alkoxy group having 1 to 6 carbon atoms, or a heterocyclic alkyl group, and
R² is a hydrogen atom, a hydroxyl group, an alkyl group having 1 to 6 carbon atoms, or an alkoxy group having 1 to 6 carbon atoms, or R¹ and R² together form a 5 to 7 membered heterocyclic ring, which may have a substituent, said substituent being a hydroxyl group, an alkyl group having 1 to 6 carbon atoms, an alkoxy group having 1 to 6 carbon atoms, or a phenylalkyl group having 6 to 12 carbon atoms,
R³ is a hydrogen atom, a hydroxyl group, an alkyl group having 1 to 6 carbon atoms, or an alkoxy group having 1 to 6 carbon atoms, or a salt thereof, or
a compound represented by a following formula (2) or (3): 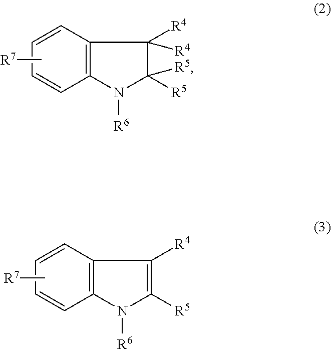
wherein R⁴ is each independently a hydrogen atom, an aldehyde group, a hydroxy group, an alkyl group having 1 to 6 carbon atoms, or an alkyl ester alkylene group, or two R⁴ together are an oxygen atom,
R⁵ is each independently a hydrogen atom, an aldehyde group, a hydroxy group, an alkyl group having 1 to 6 carbon atoms, an alkyl ester group having 1 to 6 carbon atoms, or an alkyl ester alkylene group, or two R⁵ together are an oxygen atom,
R⁶ is a hydrogen atom or an alkyl group having 1 to 6 carbon atoms, and
R⁷ is a hydrogen atom, a hydroxyl group, an alkyl group having 1 to 6 carbon atoms, an alkyl ester group having 1 to 6 carbon atoms, or an alkoxy group having 1 to 6 carbon atoms, or a salt thereof, to a patient having prostate cancer.

2. The method for treating prostate cancer according to claim 1, wherein the compound represented by the formula (1) is a compound selected from the group consisting of 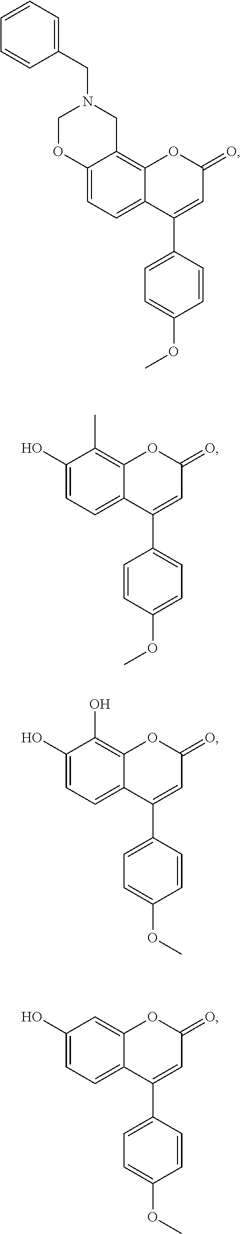 [SMILES: COc1ccc(-c2cc(=O)oc3c4c(ccc23)OCN(Cc2ccccc2)C4)cc1 ; COc1ccc(-c2cc(=O)oc3c(C)c(O)ccc23)cc1 ; COc1ccc(-c2cc(=O)oc3c(O)c(O)ccc23)cc1 ; COc1ccc(-c2cc(=O)oc3cc(O)ccc23)cc1] 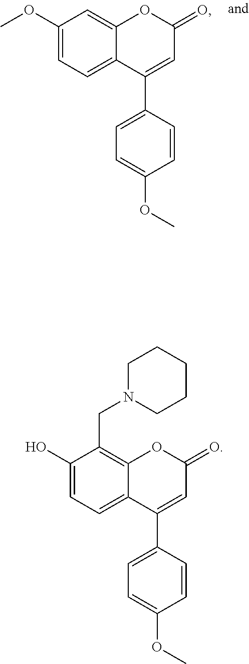 [SMILES: COc1ccc(-c2cc(=O)oc3cc(OC)ccc23)cc1 ; COc1ccc(-c2cc(=O)oc3c(CN4CCCCC4)c(O)ccc23)cc1]
the compound represented by the formula (2) is a compound selected from the group consisting of

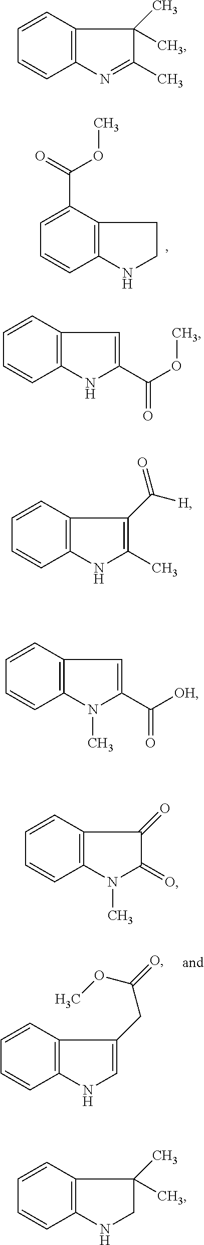

3. A method for treating breast cancer comprising: a step of administering a therapeutically effective amount of a compound represented by a following formula (1): 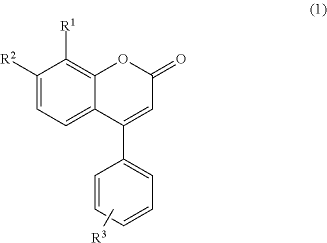
wherein R¹ is a hydrogen atom, a hydroxyl group, an alkyl group having 1 to 6 carbon atoms, an alkoxy group having 1 to 6 carbon atoms, or a heterocyclic alkyl group, and
R² is a hydrogen atom, a hydroxyl group, an alkyl group having 1 to 6 carbon atoms, or an alkoxy group having 1 to 6 carbon atoms, or R¹ and R² together form a 5 to 7 membered heterocyclic ring, which may have a substituent, said substituent being a hydroxyl group, an alkyl group having 1 to 6 carbon atoms, an alkoxy group having 1 to 6 carbon atoms, or a phenylalkyl group having 6 to 12 carbon atoms,
R³ is a hydrogen atom, a hydroxyl group, an alkyl group having 1 to 6 carbon atoms, or an alkoxy group having 1 to 6 carbon atoms, or a salt thereof, or a compound represented by a following formula (2) or (3): 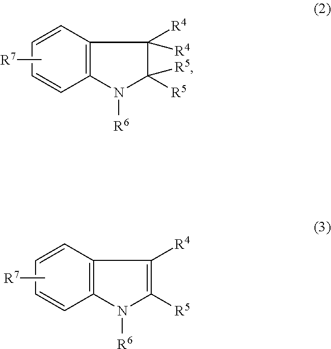
wherein R⁴ is each independently a hydrogen atom, an aldehyde group, a hydroxy group, an alkyl group having 1 to 6 carbon atoms, or an alkyl ester alkylene group, or two R⁴ together are an oxygen atom,
R⁵ is each independently a hydrogen atom, an aldehyde group, a hydroxy group, an alkyl group having 1 to 6 carbon atoms, an alkyl ester group having 1 to 6 carbon atoms, or an alkyl ester alkylene group, or two R⁵ together are an oxygen atom,
R⁶ is a hydrogen atom or an alkyl group having 1 to 6 carbon atoms, and
R⁷ is a hydrogen atom, a hydroxyl group, an alkyl group having 1 to 6 carbon atoms, an alkyl ester group having 1 to 6 carbon atoms, or an alkoxy group having 1 to 6 carbon atoms, or a salt thereof, as an active ingredient.

4. The method for treating breast cancer according to claim 3, wherein the compound represented by the formula (1) is a compound selected from the group consisting of 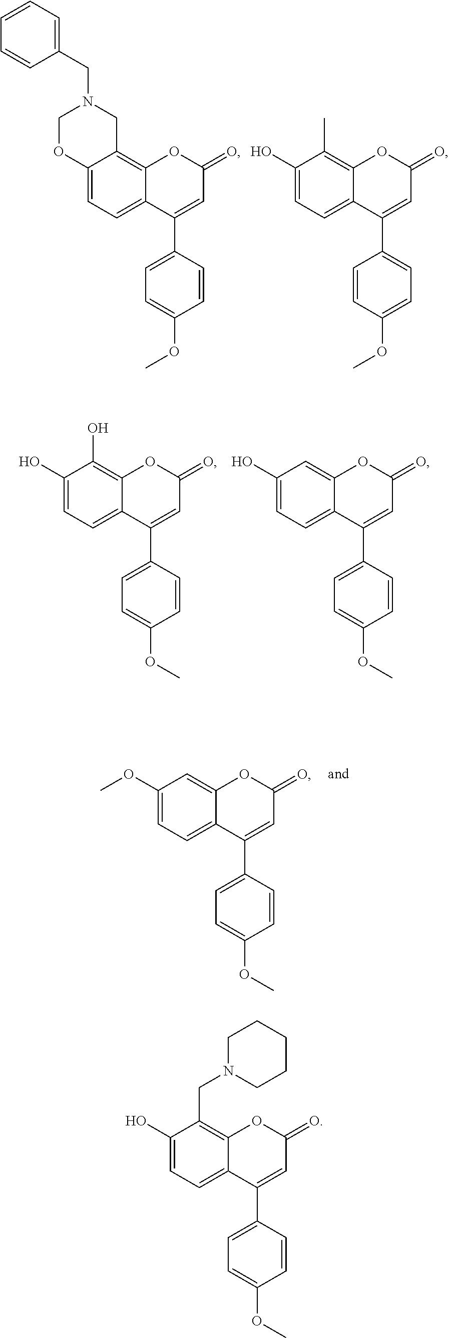
the compound represented by the formula (2) is a compound selected from the group consisting of

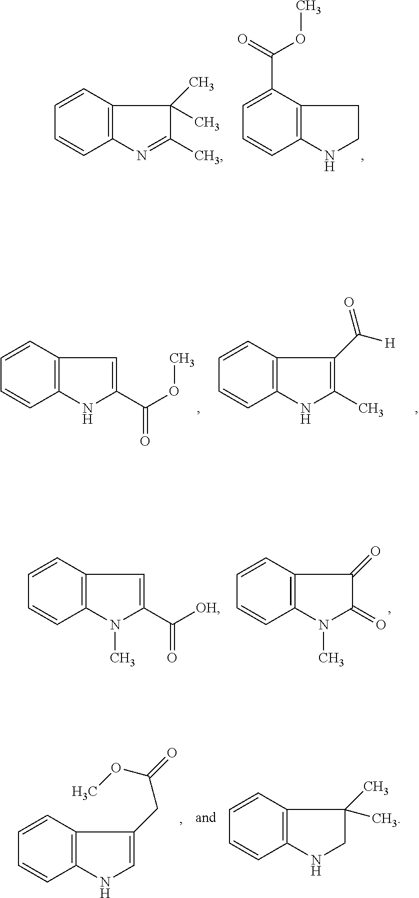

5. The method for treating prostate cancer according to claim 1, wherein the compound represented by the formula (1) is the following compound:

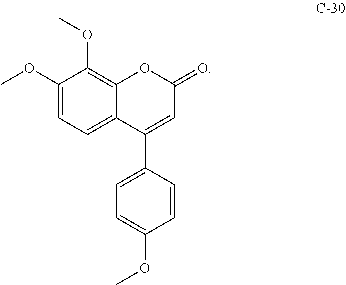 [SMILES: COc1ccc(-c2cc(=O)oc3c(OC)c(OC)ccc23)cc1]

6. The method for treating breast cancer according to claim 3, wherein the compound represented by the formula (1) is the following compound:

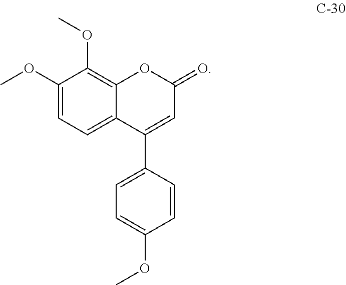 [SMILES: COc1ccc(-c2cc(=O)oc3c(OC)c(OC)ccc23)cc1]

7. A method for treating prostate cancer or breast cancer comprising: a step of administering a therapeutically effective amount of a compound represented by any one of the following formulae: 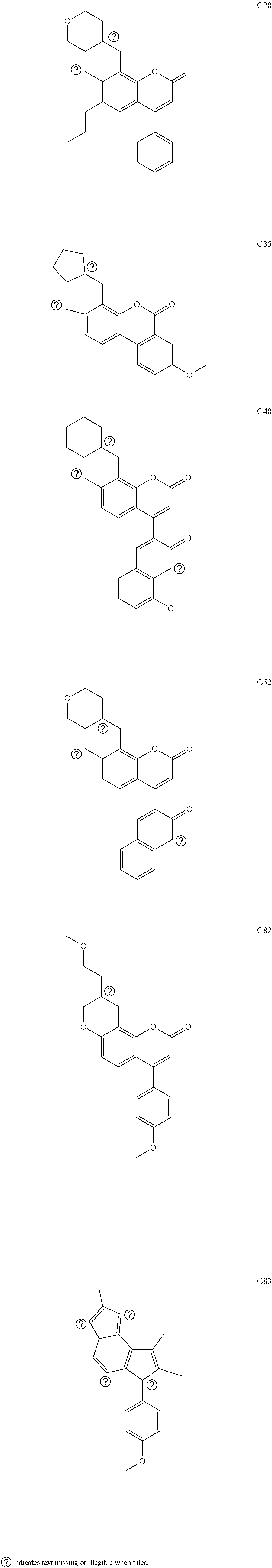
or a salt thereof, to a patient having prostate cancer or breast cancer.
